(19) United States
(12) Patent Application Publication
(10) Pub. No.: US 2008/0086136 A1
(43) Pub. Date: Apr. 10, 2008

(54) Percutaneous hip system

(76) Inventor: Drew A. Bednar, Hamilton (CA)

Correspondence Address: OGILVY RENAULT LLP, 1981 MCGILL COLLEGE AVENUE, SUITE 1600, MONTREAL, QC, H3A2Y3, US

(21) Appl. No.: 11/512,231

(22) Filed: Aug. 30, 2006

(51) Int. Cl. A61F 2/30

(52) U.S. Cl. 606/69

(57) ABSTRACT

A kit for fixation of a fracture in a superior portion of a femoral bone, to minimize patient trauma by minimizing the incision during surgery, the kit having: a tube having a distal end and a proximal end, and a tube bore aligned with a longitudinal tube axis, the tube bore having a maximum internal dimension in a plane transverse to the tube axis; and a bone plate having a barrel portion with a barrel bore aligned with a barrel axis and a bone-engaging portion disposed at a selected angle to the barrel axis, the bone plate having an external dimension, in a plane transverse to the barrel axis, less than the maximum internal dimension of the tube bore.

### TECHNICAL FIELD

The invention relates to percutaneous bone fracture fixation in the superior portion of the femoral bone.

### BACKGROUND OF THE ART

Fixation of fractures in the superior portion of the femoral bone typically involves introducing a lag screw across the fracture and a bone plate to hold the lag screw and fracture in place. Bone screws pass through the bone plate and fix the plate to an intact portion of the bone.

The prior art procedure typically requires a relatively large incision to be made down the hip and leg of the patient, in order to gain access to the femoral bone and introduce the fixation devices. Obese patients may require much larger incisions due to thicker fat layers. Such a large incision is associated with prolonged pain and hospital stay for the patient, as well as a greater possibility of developing other comorbidities. In addition, patients requiring treatment for such fractures are typically older less mobile adults with slower rates of healing. Thus, such a large incision will mean extensive pain and bed rest for the patient, causing a significant negative impact on the patient's quality of life, which may last for an extended period of time.

Prior art devices have attempted to minimize this negative impact by providing smaller bone plates for treating a femoral fracture, such as disclosed in U.S. Application Publication 2004/0193162 and U.S. Pat. No. 2,397,545. While such devices might decrease the length of the necessary incision, a substantial incision is still required to access the bone, and perform the procedure.

Percutaneous treatment in certain orthopedic procedures is generally preferred since the incision and accompanying recuperation are significantly lessened. These include the use of an access tube to percutaneously treat and introduce devices to the knee, as in U.S. Application Publication 2004/0243138, and the use of an access tube to remove orthopedic screws, as in U.S. Application Publication 2004/0158257.

Features that distinguish the present invention from the background art will be apparent from review of the disclosure, drawings and description of the invention presented below.

### DISCLOSURE OF THE INVENTION

A first embodiment of the present invention provides a kit used for percutaneous fixation of a femoral fracture. In accordance with the first embodiment of the present invention, there is a kit for fixation of a fracture in a superior portion of a femoral bone, to minimize patient trauma by minimizing the incision during surgery, the kit having: a tube having a distal end and a proximal end, and a tube bore aligned with a longitudinal tube axis, the tube bore having a maximum internal dimension in a plane transverse to the tube axis; and a bone plate having a barrel portion with a barrel bore aligned with a barrel axis and a bone-engaging portion disposed at a selected angle to the barrel axis, the bone plate having an external dimension, in a plane transverse to the barrel axis, less than the maximum internal dimension of the tube bore.

Preferably, the tube is small enough to be introduced into the patient through a minimal incision, and large enough to provide passage for devices commonly used to treat femoral fractures.

Another embodiment of the invention provides a percutaneous method for fixation of a fracture in the superior portion of a femoral bone using a tube and a bone plate designed to pass through said tube, minimizing the size of an incision.

Further aspects of the embodiments will become apparent upon reference to the accompanying drawings and description.

### DESCRIPTION OF THE DRAWINGS

In order that the invention may be readily understood, embodiments of the invention are illustrated by way of example in the accompanying drawings.

FIG. 1 depicts a set of coaxial nested tubes to guide a central fastener or threaded wire being applied to a femoral bone.

FIG. 2A is an exploded perspective view of the tubes of FIG. 1.

FIG. 2B is an exploded perspective view of an alignment guide used with the largest tube.

FIG. 3 depicts the alignment guide used to install parallel wire fasteners to a femoral bone.

FIG. 4 depicts another embodiment of the tube having a handle grip showing a larger blind hole drilled about the central wire fastener.

FIG. 5 is a perspective view of the tube of FIG. 4.

FIG. 6 depicts a bone plate and fasteners being applied to the femoral bone.

FIG. 7 is a close-up perspective view of the barrel of the bone plate with an optional keyed internal profile in alignment with a lag screw end having a matching keyed external profile to prevent rotation.

FIG. 8 is a close-up side view of a cross-section of the inferior end of the bone plate, showing a bone screw fitting into the bone plate.

FIG. 9 is a perspective view of elongate appendages and a view of the appendages introducing a medical substance sponge into a blind hole drilled in the femoral bone.

FIG. 10 is a close-up view of the appendages of FIG. 9 gripping the medical substance sponge.

FIG. 11 is a cut-out of the appendages of FIG. 10 along line 11-11 showing optional appendage protrusions.

Further details of the invention and its advantages will be apparent from the detailed description included below.

### DETAILED DESCRIPTION OF PREFERRED EMBODIMENTS

The illustrated embodiment of the present invention is a kit including a tube 5, a bone plate 26, a lag screw 24. Optionally a compression screw 37, bone screws 35, an alignment guide 13, and elongate appendages 38 may be included. The details and use of these parts will be described below with reference to the drawings by way of example.

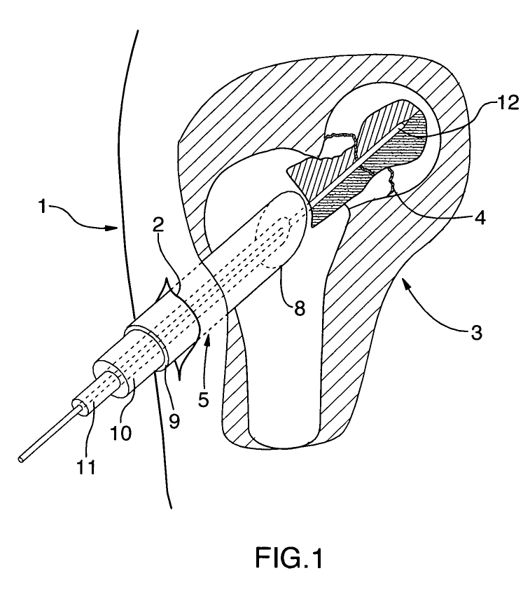

FIG. 1 shows the present invention as applied to a patient 1 through an incision 2, against the femoral bone 3, which has a fracture 4 in the superior portion. The tube 5 may include a plurality of different sized nested and coaxial portions 10 and 11. The distal end of the tube 5 is introduced to the bone 3 through the incision 2, and is guided into position by loading the tube 5 over a conventional K-wire 12.

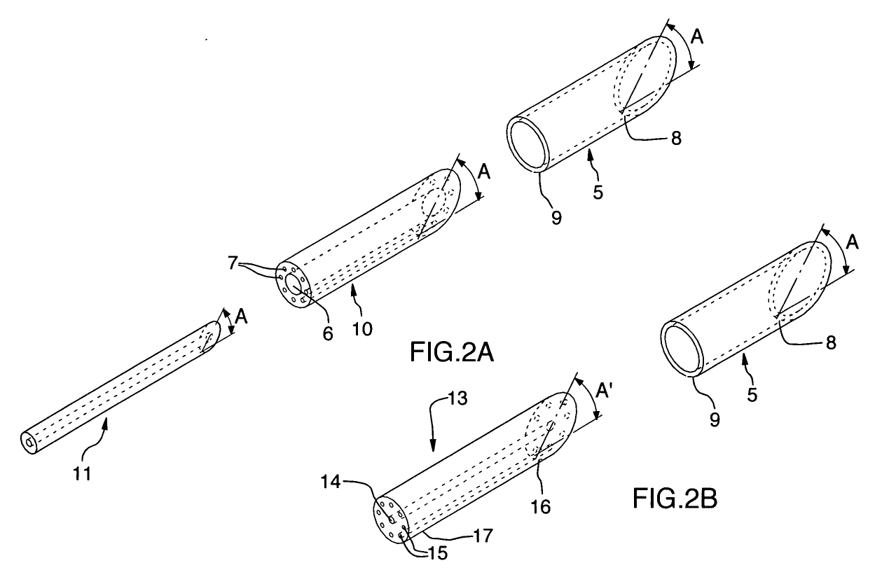

The incision 2 is small initially and accommodates only the smallest tube 11. The incision 2 is then dilated to the size necessary for the procedure, by introducing tubes 10 and 5 of increasing size, as shown in FIG. 1, or by other means, such as an expanding tube, not shown. Regardless of the exact method, the tube 5 is introduced to the bone 3 to provide a passage sufficiently large to treat the fracture 4, through a small incision 2 to minimize patient trauma. Further shown in FIG. 1 is that tube 5 has a distal end 8 and a proximal end 9 seen more clearly in FIG. 2.

In a preferred embodiment, dilation of the incision 2 is performed by telescoping tubes 5, 11, and 10, which are all cylindrical in the illustrations but need not be ,imited to this configuration. FIG. 2 shows the telescoping tubes 5, 11 and 10 separately. Considering only the largest tube 5, it can be seen that the distal end 8 is distinct from the proximal end 9 in that distal end 8 is angled relative to the longitudinal axis whereas the proximal end 9 is normal to the longitudinal axis. The distal end 8 is directed towards the bone 3, and has an angle to aid in correctly directing the procedure. The tubes 5, 11 and 10 have differing inner and outer diameters to allow them to nest together. Each of the tubes 5, 11 and 10 has distinct distal ends 8 and proximal ends 9, having the same angle on the distal end 8.

In a preferred embodiment, the smallest tube 11 has a length of 150 mm, inner diameter of 2.3 mm to fit over the K-wire 12, and an outer diameter of 15 mm, such that the initial incision 2 causes minimal patient trauma. The middle tube 10 has a length of 150 mm, an inner diameter of 15 mm to fit over the smallest tube 11, and an outer diameter of 30 mm. The largest tube 5 has a length of 150 mm, an inner diameter of 30 mm to fit over the middle tube, and an outer diameter of 40 mm. The distal end 8 of each tube has a preferred angle A (FIG. 2) of 45°. It will be appreciated that tubes 10 and 11 are simply variations on tube 5 and are included in any description referring to tube 5.

As shown in FIG. 2, in the preferred embodiment, the middle tube 10 has a centered bore 6 and evenly spaced offset channels 7 circumferentially located about the bore 6. There are preferably eight such channels 7, each having an inner diameter of 2.8 mm, circumferentially located on a circle of diameter 20 mm to allow passage of other devices useful for the procedure.

Also shown in FIG. 2 is an alignment guide 13, designed to fit into the largest tube 5, and replacing the smaller tubes 10 and 11. The alignment guide preferably has a length of 150 mm, a centered bore 14 with an inner diameter of 2.3 mm to fit over the K-wire 12, and an outer diameter of 30 mm to fit inside the largest tube 5. Because the alignment guide 13 replaces middle tube 10, the alignment guide 13 also has offset channels 15, shown here to have the same arrangement as on middle tube 10, but other arrangements are possible. The alignment guide has distal end 16 and proximal end 17, where the distal end 16 is directed towards the bone 3. The distal end 16 of the alignment guide 13 is angled, but may differ from the angle on the tube 5. In a preferred embodiment, the alignment guide 13 is angled at an angle A′ of 30°-40°, allowing a range of access orientations for performing the procedure. There may be a plurality of alignment guides, each providing a different angle at the distal end 16 or providing a different arrangement of the offset channels 15.

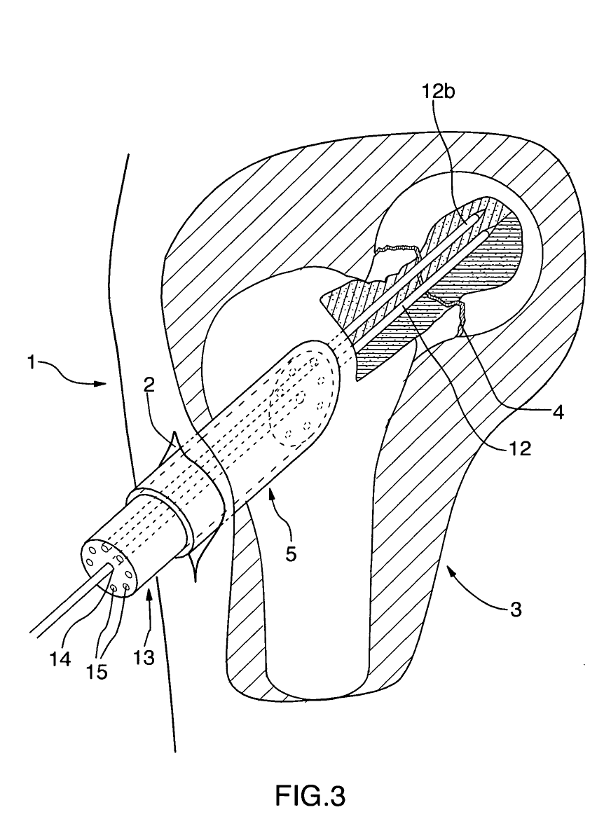

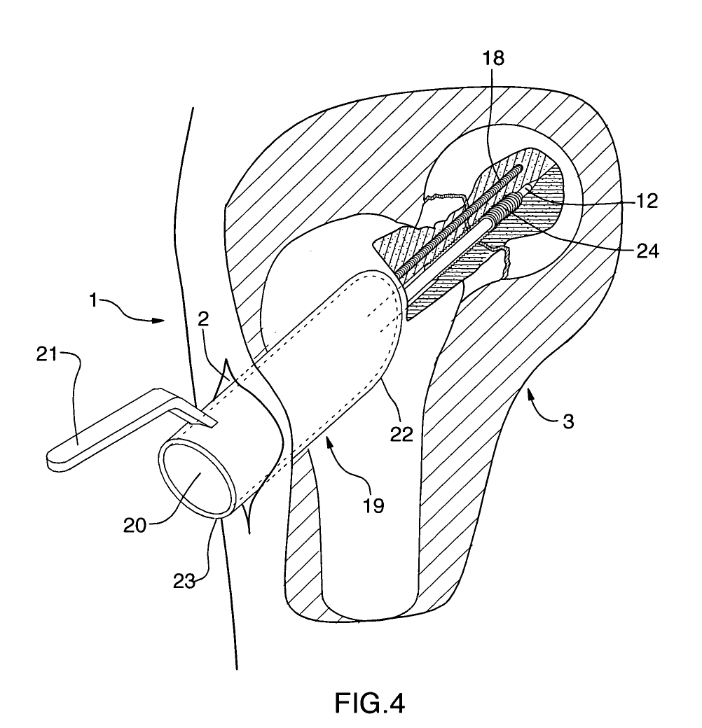

FIG. 3 shows how alignment guide 13 might be used with tube 5 in the procedure. A second K-wire 12b may be introduced to the bone 3 through one of the offset channels 15 on the alignment guide 13. The alignment guide 13 can now be removed and the second K-wire 12b can be used to guide a derotation screw 18 (FIG. 4) into the bone. A derotation screw 18 is commonly used to fix fractures in a femoral bone, and is commonly introduced at the end of the procedure. However, it will be appreciated by persons skilled in the art that the derotation screw 18 may be introduced near the start of the procedure, and may aid in fixing the fracture 4 by preventing motion in the fracture 4 in subsequent steps in the procedure.

Once the incision 2 has been dilated and the derotation screw 18, if using, has been introduced across the fracture 4, an access tube 19 can be introduced over the largest tube 5. It will be appreciated that the access tube 19 is simply another variation of tube 5 and is included in any description referring to the tube 5. Tube 5 is removed from the access tube 19 along with other tubes 10, 11, or alignment tube 13 if these are still in place, thus providing an open passage for introducing the lag screw 24. The lag screw 24 preferably has a bone adhesion promotion coating on its outer surface, such as hydroxyapatite. A preferred embodiment of the access tube 19 has a handle 21 to aid in positioning the tube, a distal end 22 and a proximal end 23.

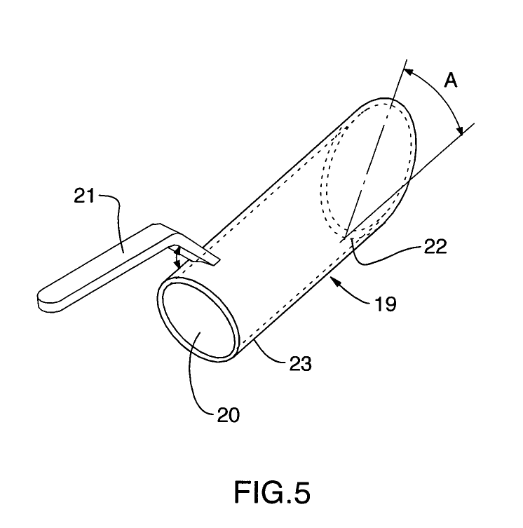

Details of the access tube 19 can be better appreciated in FIG. 5. A preferred embodiment of the access tube 19 has a length of 100-150 mm, an inner diameter of 40 mm to fit over the largest tube 5, and a wall thickness of 1 mm, which maximizes the diameter of the access passage without significantly increasing the size of the incision 2. The distal end 22 is angled at the same angle A as tubes 5, 10, and 11, the angle being 45° in the preferred embodiment. The handle 19 branches off from the proximal end 23 at a preferred angle of 60° to aid in positioning the access tube 19 and in holding it in place against the bone 3. The access tube 19 is preferably made of a biocompatible, radiolucent material, such as an aluminum alloy.

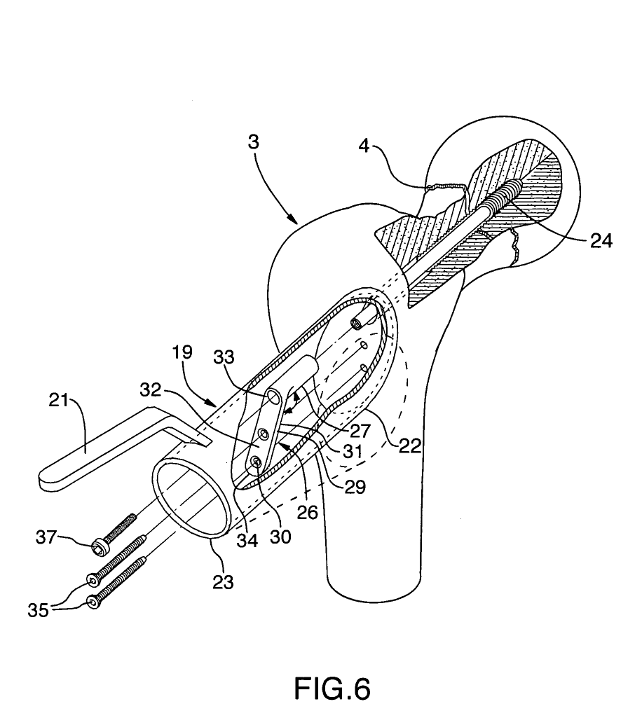

Once the access tube 19 is in place and the lag screw 24 has been introduced across the fracture 4, bone plate 26 can be introduced to the bone 3, as shown in FIG. 6. An elongated cylinder or rod, not shown, can be used to advance the bone plate 26 through the access tube 19 towards the bone. The bone plate 26 has a barrel portion 27 and a bone-engaging portion 29. The bone-engaging portion has a bone-engaging medial side 31 and an opposite lateral side 32. The bone-engaging portion also has a superior end 33 and an opposite inferior end 34. In a preferred embodiment, the barrel portion 27 and the bone-engaging portion 29 form a selected angle of 135°, matching the angle on the access tube distal end 22. There may be a plurality of bone plates 26 with selected angles ranging from 140°-150°, to match the angle on the alignment guide distal end 16. In a preferred embodiment, the bone plate 26 is designed to pass through the access tube 19 when the axis of the barrel portion 27 is parallel to the longitudinal axis of the access tube 19. Referring to FIGS. 6 and 8, the bone-engaging inferior end 34 has a rounded end and is beveled at angle A, matching the angle of the access tube 19, to better allow bone plate 26 to pass through the access tube 19 without interference.

As shown by the dotted oval in FIG. 6, the access tube 19 can be pivoted about its proximal end 23 in order to provide access to different locations on the bone 3 without having to increase the size of the incision 2. This allows different areas of the bone plate 26 to be centered in the access tube 19, for example to aid in fitting bone screws 35 into the screw holes 30.

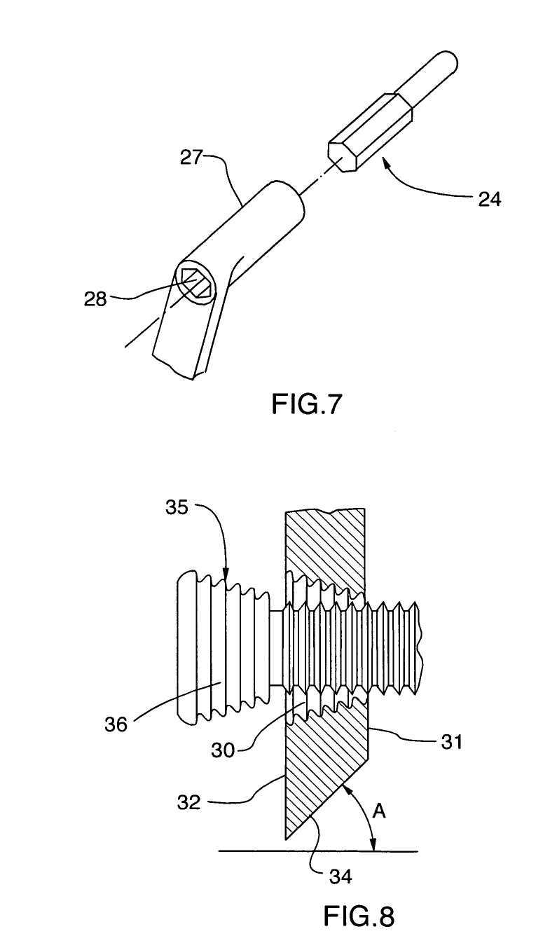

In one embodiment, the barrel portion 27 is free to rotate about the lag screw 24, to ease installation of the barrel 27 on the lag screw 24, and ease passage of the bone plate 26 through the access tube 19. However, the barrel portion 27 can also be designed to prevent rotation of the bone plate 26 about the lag screw 24. This can be done for example by having a keyed internal profile 28 that is non-circular and matching the keyed outer profile of the lag screw 24, as shown in FIG. 7.

The bone plate 26 is introduced to the bone 3 and the barrel portion 27 is loaded over the lag screw 24. The bone-engaging portion 29 is then attached to the bone 3 preferably using bone screws 35. This attachment, as with all steps in the procedure, is performed percutaneously through a tube, such as access tube 19. It will be appreciated by persons skilled in the art that the bone-engaging portion 29 can be attached to bone 3 using other fastening means, such as bone wires or bone staples, and that the bone plate 26 can be modified to be compatible with these other fastening means without deviating from the spirit of the present invention. In the preferred embodiment, the bone screws 35 fit into screw holes 30 on the bone plate 29. The screw holes 30 may be tapered towards the medial side 31, and may be threaded, as shown in FIG. 8. The bone screws 35 may also be threaded and tapered to match the screw holes 30, to maximize attachment of the bone plate 29 to the bone 3 and prevent future loosening.

A compression screw 37 can be introduced to compress the bone fracture as the barrel 33 of the bone plate 29 is drawn longitudinally towards the distal embedded end of the lag screw 24, thus closing up fracture 4 even further. Once the fracture 4 is closed and stabilized, the access tube 19 can be removed from the patient 1 and the incision 2 can be closed.

An optional step in the procedure is shown in FIG. 9. Elongate appendages 38 are used to introduce a medical substance 42, shown here on a sponge carrier, into the blind hole drilled into the bone 3 to aid in fixation or healing of the fracture 4. The medical substance 42 is introduced over the K-wire 12 after the bone 3 has been drilled but before the introduction of the lag screw 24.

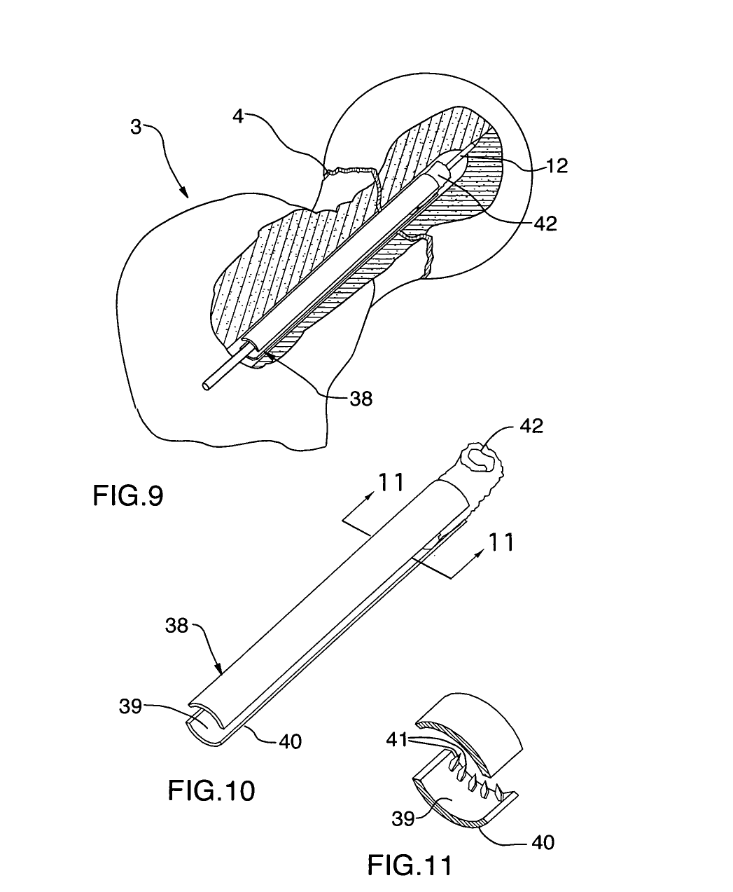

As shown in greater detail in FIG. 10, the preferred embodiment, comprises two elongate appendages 38. The appendages 38 each have a concave inner surface 39 and a convex outer surface 40 curved about their longitudinal axis. FIG. 11 shows a cutout of one of the appendages 38 along line 11-11. The cutout reveals the inner surface 39, which preferably has protruding grips 41 to aid in gripping and pushing the medical substance 42. The medical substance 42 may be an osteoinductive agent or any other medical substance introduced to the bone in a fracture fixation procedure. A medical substance 42 may also be introduced into the bone by other means through the access tube 19. For example, an angiogenic biological or a bone cement may be injected into the bone to aid in fracture healing or fixation.

Although the above description relates to a specific preferred embodiment as presently contemplated by the inventor, it will be understood that the invention in its broad aspect includes mechanical and functional equivalents of the elements described herein.

I claim:

1. A kit for fixation of a fracture in a superior portion of a femoral bone comprising:
a) a tube having a distal end and a proximal end, and a tube bore aligned with a longitudinal tube axis, the tube bore having a maximum internal dimension in a plane transverse to the tube axis; and
b) a bone plate having a barrel portion with a barrel bore aligned with a barrel axis and a bone-engaging portion disposed at a selected angle to the barrel axis, the bone plate having an external dimension, in a plane transverse to the barrel axis, less than the maximum internal dimension of the tube bore.

2. The kit of claim 1 also comprising at least one fastener engagable with the bone-engaging portion of the bone plate and the femoral bone.

3. The kit of claim 2 wherein the fastener is selected from the group consisting of: a bone screw; a wire; and a bone staple.

4. The kit of claim 1 also comprising a lag screw having a proximal end mating the barrel bore and a distal end with bone engagement threads.

5. The kit of claim 4 wherein an outer surface of the lag screw has a bone adhesion promotion coating.

6. The kit of claim 1 comprising a plurality of said tubes, each having a different cross-section.

7. The kit of claim 1 wherein the tube has an angled distal end matching the selected angle of the bone plate.

8. The kit of claim 1 wherein the tube has a handle adjacent the proximal end.

9. The kit of claim 1 wherein an inferior end of the bone-engaging portion of the bone plate has a profile matching at least a segment of the tube bore.

10. The kit of claim 2 wherein the bone-engaging portion of the bone plate has at least one bone screw hole.

11. The kit of claim 10 wherein the bone screw hole is internally threaded and tapered towards a medial side of the bone plate, and wherein a matching bone screw has a tapered head and is externally threaded.

12. The kit of claim 1 wherein the tube has at least one channel parallel to the tube axis and offset from the tube bore.

13. The kit of claim 12 wherein the tube has a plurality of said channels circumferentially spaced about the tube bore.

14. The kit of claim 4 wherein the lag screw has a keyed cross-sectional profile that mates with a rotation-preventing keyed internal profile in the barrel bore.

15. The kit of claim 4 comprising a compression screw engaging the barrel of the bone plate and the lag screw adjacent the proximal end thereof.

16. The kit of claim 1 comprising:
an alignment guide, engageable with the tube, having a guide bore along a longitudinal guide axis and at least one channel parallel to and offset from said guide bore.

17. The kit of claim 1 comprising at least two elongate appendages each having an outer surface matching the bore of the tube.

18. The kit of claim 17 wherein the appendages have an inner surface having protruding grips.

19. A method for fixation of a fracture in the superior portion of a femoral bone comprising:
a) gaining access to the bone through an incision of length sufficient to insert a tube with a bore along its longitudinal axis, said tube having a distal end and a proximal end;
b) through the tube bore, passing a lag screw and embedding the lag screw in a blind hole drilled across the fracture;
c) through the tube bore, passing a bone plate having a barrel portion with a barrel bore aligned with a barrel axis and a bone-engaging portion disposed at a selected angle to the barrel axis, the bone plate having an external dimension, in a plane transverse to the barrel axis, less than the maximum internal dimension of the tube bore; and
d) through the tube bore, assembling the lag screw in the barrel of the bone plate, and attaching the bone plate onto the bone with at least one fastener.

20. The method of claim 19 further comprising drilling a blind hole in the bone across the fracture after gaining access to the bone and before embedding either one of a K-wire and a lag screw into the bone.

21. The method of claim 19 further comprising manipulation of the distal end of the tube within the incision to access different portions of the bone through the proximal end of the tube bore.

22. The method of claim 19 further comprising introducing a medical substance into the blind hole in the bone before the assembling step.

23. The method of claim 22 wherein the medical substance is selected from the group consisting of: an osteoinductive agent, a bone cement, and an angiogenic biological.

24. The method of claim 19 further wherein said tube has at least one channel parallel to the bore and offset from the bore, and wherein the method comprises:
introducing at least one of: the lag screw; the fastener; and a treatment device, through the channel of said tube.
